(19) United States
(12) Patent Application Publication
(10) Pub. No.: US 2002/0102618 A1
(43) Pub. Date: Aug. 1, 2002

(54) DROSOPHILA DERIVED RECEPTORS OF THE STEROID RECEPTOR SUPERFAMILY

(76) Inventors: RONALD M. EVANS, LA JOLLA, CA (US); ESTELITA S. ONG, SAN DIEGO, CA (US); ANTHONY E. ORO, SAN DIEGO, CA (US)

Correspondence Address: STEPHEN E. REITER, FOLEY & LARDNER, P.O. BOX 80278, SAN DIEGO, CA, 92138-0278, US

(21) Appl. No.: 08/425,716

(22) Filed: Apr. 19, 1995

Related U.S. Application Data
(63) Continuation of application No. 07/979,479, filed on Nov. 20, 1992, now abandoned.
Application No. 07/979,479 is a continuation of application No. 07/734,051, filed on Jul. 22, 1991, now abandoned.
Application No. 07/734,051 is a division of application No. 07/278,606, filed on Nov. 30, 1988, now abandoned.

(51) Int. Cl. G01N 33/53; G01N 33/567

(52) U.S. Cl. 435/7.21; 435/7.2

(57) ABSTRACT

Disclosed is an identified model Drosophila melanogaster receptor, characterized and described for potential use for identifying other related polypeptides and for use in assays to screen and develop potential anthelmintics, for example.

### FIELD OF THE INVENTION

This invention relates generally to the field of molecular biology and to certain advances made in this field, and particularly, to research directed to the identification, characterization and use of certain insect receptor polypeptides, paving the way for the production of new anthelmintics, such as insecticides, for example.

### BACKGROUND OF THE INVENTION

The last decade or so has witnessed enormous research efforts in the field of molecular biology. Many of these efforts centered initially on studies with Drosophila genes, primarily because of their relatively rapid turnover in successive generations so that genetic altering of such genes could be rapidly studied as to physiological effect.

Further advances in the field of molecular biology have provided the basis for a branching of the science into different species; indeed, certain human protein products made by recombinant DNA technology are currently enjoying prominent marketing status.

Recent research efforts have reemerged in the form of studies of DNA of the Drosophila genus. The present invention is based in part upon such research.

The goal has been to gain an understanding of the mechanisms involved in various receptor polypeptides functioning in various organisms. The present invention is based upon the identification of novel isolate receptor DNA and a consequent polypeptide product produced by application of recombinant DNA technology involving the expression of the DNA in a transfected host organism.

The present invention shall find use in the development of assays utilizing the novel Drosophila receptor hereof in the screening of extrinsic materials that may have a modulating effect on said receptor, thus paving the way for the screening, characterization, and development of certain materials that meet the criterion of having a certain, desirable modulating effect on a Drosophila receptor or related molecule, for example, so as to be useful in the preparation of various anthelmintic compositions, e.g., insecticides.

It is an object of the present invention to screen, identify, characterize and produce, particularly via recombinant DNA technology, extrinsic materials that may have a certain modulating effect upon an insect receptor, so as to be useful for the development of such materials per se or as suitable compositions for use as anthelmintics, for example.

### SUMMARY OF THE INVENTION

The present invention is predicated upon the identification and isolation of sufficient quality and quantity of a model Drosophila receptor polypeptide that has enabled the discriminate characterization thereof, both in terms of physical attributes and their biological function and effect. These results have enabled in turn the consequence that insect receptors can be employed in a method for screening extrinsic materials that may modulate its activity which comprises challenging the said receptor species or functional fragment thereof with one or more of a battery of test materials that can potentially modulate the biofunction of said receptor and monitoring the effect of said material on said receptor in an in vitro setting.

The present invention is further directed to an expression vector capable of producing an insect receptor or functional fragment thereof which comprises expression control elements operative in the recombinant host selected for the expression of DNA encoding said insect receptor or functional fragment.

The invention is further directed to a DNA molecule which is a recombinant DNA molecule or a cDNA molecule consisting of a sequence encoding an insect receptor.

The invention is further directed to substantially pure insect receptor or a functional fragment thereof obtainable by expression of DNA encoding same in a transfected recombinant host organism.

The present invention thus embraces an insect receptor polypeptide, having a sequence characteristic of a mammalian receptor DNA-binding domain having flanking N-terminal and C-terminal sequences, and having purity sufficient to provide sufficient coding sequence to enable the production of total DNA coding sequence of said receptor or cross-hybridizing DNA of related receptors for use in the expression thereof in recombinant host cells operatively transfected with said DNA.

The present invention is directed to recombinant DNA technology and all aspects relating to the use of amino acid sequence of said model Drosophila receptor polypeptide for DNA isolates production, including cross-hybridizable isolates, devising expression vectors therefor, transfecting hosts producing therewith and methods comprising utilizing such information to devise cells or cell lines harboring genetic information sufficient for such cells or cell lines to produce said insect receptor such that they can be used as such or in assays for the identification or development of anthelmintics such as insecticides, for example.

### DETAILED DESCRIPTION OF THE INVENTION

### DESCRIPTION OF THE DRAWINGS

FIG. 1 depicts cross-hybridizing bands revealing the presence of retinoic acid receptor related sequences in D. melanogaster.

A. Low stringency hybridization using the EcoRI-SacI fragment of a hRAR cDNA shows six hybridizing EcoRI bands (lane 1) and five XhoI bands (lane 2), but no hybridizing bands at high stringency in either lane 1 or lane 2. A Drosophila genomic library was screened; one class of isolates contained the 4.5 kb EcoRI-XbaI genomic fragment subcloned in pRX4.5. pRX4.5 hybridizes to the 14 kb EcoRI band and the 11 kb XhoI band at high stringency, indicative of a unique gene.

B. Map of subclone pRX4.5 showing the smallest hybridizing fragment, an 800 bp PstI-DraI fragment, as a hatched box. Nucleotide sequencing revealed a region with significant homology to vertebrate steroid hormone receptor genes. Conceptual translation revealed a sequence characteristic of the second zinc finger of a steroid hormone receptor. Restriction enzyme sites are R=EcoRI; D=DraI; K=KpnI; S=SacI; P=PstI; B=BamHI; Bg=BglII; Xb=XbaI.

Methods: Standard molecular biology methods were used as in Ausubel et al., Current Protocols in Molecular Biology, Greene Publishing Assoc. and Wiley Interscience, New York, 1987, except that the conditions for low and high stringency hybridization follow those of Arriza et al., Science 237, 268 (1987).

FIG. 2 provides the complete nucleotide sequence of clone Imd2, isolated from a Drosophila imaginal disc library. The sequence begins with a presumptive initiator Met codon at nucleotide 1499, but also contains 2 additional downstream Met codons at nucleotides 1502 and 1529 that are less likely initiators according to the consensus translation initiation site for Drosophila [See Cavener, Nucleic Acids Res. 15, 1353 (1987)]. Multiple upstream stop codons (underlined in the 5′ region) further suggest the first Met codon as the translation start site. A stop codon beginning at nucleotide position 2640 is followed by a 3′ untranslated region of 1038 bp that contains multiple consensus polyadenylation signals (underlined in the 3′ region).

Methods: Nucleotide sequencing was by the dideoxynucleotide method, using inosine instead of guanosine in GC-rich regions. Sequence assembly was aided by the programs of Devereux et al., Nucleic Acids Research 12, 387 (1987).

FIG. 3 shows a comparison of the predicted knrl product to vertebrate steroid/thyroid hormone receptors.

A. Alignment of the DNA-binding domains of representative members of the superfamily, showing the conserved amino acids and the extensive structural similarity between knrl and kni. Note that the identity of knrl and kni extends past the conserved Gly and Met residues of the DNA-binding domain.

B. Overall structural comparison of the predicted protein sequence of knrl to other members of the steroid/thyroid hormone receptor superfamily. Comparisons of the region marked DNA are to the 66-68 amino acid DNA-binding domains and the region marked Ligand Binding are to the amino acids after the conserved Cy and Met residues of the DNA-binding domain. Since the structural similarity of knrl to the other receptors in the carboxy terminal region is not significant, no specific alignment of these regions is shown.

Methods: Comparisons used the programs of Devereux et a., Supra. Numbers indicate amino acids as detailed herein. See also Weinberger et al., Nature 324, 641 (1986), and Hollenberg et al., Nature 318, 635 (1985) for knrl, kni, hTRβ, hRAR, and hGR, respectively.

### GENERAL DEFINITIONS

The term “receptor” is used herein as a definition of the polypeptides described, based upon their having been isolated from a Drosophila embryonic DNA library using a probe from the DNA binding domain of the human retinoic acid receptor and based upon their having amino acid sequences similar to and diagnostic of all members of the steroid receptor superfamily.

Amino acid identification uses the single- and three-letter alphabets of amino acids, i.e.:

| | | | | | |
|---|---|---|---|---|---|
| Asp | D | Aspartic acid | Ile | I | Isoleucine |
| Thr | T | Threonine | Leu | L | Leucine |
| Ser | S | Serine | Tyr | Y | Tyrosine |
| Glu | E | Glutamic acid | Phe | F | Phenylalanine |
| Pro | P | Proline | His | H | Histidine |
| Gly | G | Glycine | Lys | K | Lysine |
| Ala | A | Alanine | Arg | R | Arginine |
| Cys | C | Cysteine | Trp | W | Tryptophan |
| Val | V | Valine | Gln | Q | Glutamine |
| Met | M | Methionine | Asn | N | Asparagine |

Insect receptors hereof are prepared 1) having methionine as the first amino acid (present by virtue of the ATG start signal codon insertion in front of the structural gene) or 2) where the methionine is intra- or extracellularly cleaved, having its ordinarily first amino acid, or 3) together with either its signal polypeptide or conjugated protein other than its conventional signal polypeptide, the signal polypeptide or a conjugate being specifically cleavable in an intra- or extracellular environment. In all events, the thus produced receptors, in their various forms, are recovered and purified to a level suitable for intended use. See Supra.

The “insect receptors” hereof include the specific receptors disclosed, for all species that cross-hybridization exists, as well as related (e.g., gene family) receptors that are enabled by virtue of DNA isolation and characterization and use via cross-hybridization techniques from said specific receptors or from identification via immuno cross-reactivity to antibodies raised to determinants in the usual manner known per se. It also includes functional equivalents of all of the above, differing in one or more amino acids from the corresponding parental (wild-type) species, or in glycosylation and/or phosphorylation patterns, or in bounded conformational structure.

“Expression vector” includes vectors which are capable of expressing DNA sequences contained therein, where such sequences are operatively linked to other sequences capable of effecting their expression. It is implied, although not always explicitly stated, that these expression vectors may be replicable in the host organisms either as episomes or as an integral part of the chromosomal DNA. “Operative,” or grammatical equivalents, means that the respective DNA sequences are operational, that is, work for their intended purposes. In sum, “expression vector” is given a functional definition, and any DNA sequence which is capable of effecting expression of a specified DNA sequence disposed therein is included in this term as it is applied to the specified sequence. In general, expression vectors of utility in recombinant DNA techniques are often in the form of “plasmids” which refer to circular double stranded DNA loops which, in their vector form, are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

“Recombinant host cells” refers to cells which have been transfected with vectors constructed using recombinant DNA techniques.

“Extrinsic support medium” includes those known or devised media that can support the cells in a growth phase or maintain them in a viable state such that they can perform their recombinantly harnessed function. See, for example, ATCC Media Handbook, Ed. Cote et al., American Type Culture Collection, Rockville, Md. (1984). A growth supporting medium for mammalian cells, for example, preferably contains a serum supplement such as fetal calf serum or other supplementing component commonly used to facilitate cell growth and division such as hydrolysates of animal meat or milk, tissue or organ extracts, macerated clots or their extracts, and so forth. Other suitable medium components include, for example, transferrin, insulin and various metals.

The vectors and methods disclosed herein are suitable for use in host cells over a wide range of prokaryotic and eukaryotic organisms.

In addition to the above discussion and the various references to existing literature teachings, reference is made to standard textbooks of molecular biology that contain definitions and methods and means for carrying out basic techniques encompassed by the present invention. See for example Maniatis et al., Molecular Cloning: A Laboratory Manual, Cold Spring Harbor Laboratory, New York, 1982, and the various references cited therein, and in particular, Colowick et al., Methods in Enzymology 52, Academic Press, Inc., 1987. All of the herein cited publications are by this reference hereby expressly incorporated herein.

The foregoing description and following experimental details set forth the methodology employed initially by the present researchers in identifying and isolating a particular Drosophila receptor. The art skilled will recognize that by supplying the present information including the DNA and polypeptide sequences, and characterization and use of these receptors, as detailed herein, it is not necessary, or perhaps even scientifically advisable, to repeat these details in their endeavors to reproduce this work. Instead, they may choose to employ alternative reliable and known methods. Thus, for example, they may synthesize the underlying DNA sequences for deployment within similar or other suitable, operative expression vectors and culture systems. They may use the sequences herein to create probes, preferably from regions at both the N-terminus and C-terminus, to screen genomic libraries in isolating total encoding DNA for employment as described above. They may use the sequence information herein in cross-hybridization procedures to isolate, characterize and deploy as above-described, DNA encoding receptors of various species, or DNA encoding related (e.g., gene family) receptors or fragments thereof of the same or other species, or to devise DNA for such characterization, use and deployment encoding functionally equivalent receptors or fragments thereof of all of the above differing in one or more amino acids from parental (wild-type) species or glycosylation and/or phosphorylation patterns or in bounded conformational structure.

Thus in addition to supplying details actually employed, the present disclosure serves to enable reproduction of the specific receptor disclosed and others, and fragments thereof, using means within the skill of the art having benefit of the present disclosure. All of such means are included within the enablement and scope of the present invention.

The following examples detail materials and methods employed in the experimental procedures that follow:

### EXAMPLES

A Drosophila genomic library was screened for steroid receptor homologs with a human retinoic acid receptor (hRAR) cDNA as a hybridization probe. See Giguere et al., Nature 330, 624 (1987) and Petkovich et al., Nature 330, 444 (1987). Of several clones recovered, one mapped to chromosomal position 77E1-2, the cytological location of the gap segmentation gene knirps (kni), [Nusslian-Volhard, Nature 287, 795 (1980)]. Sequence analysis of a cDNA clone representing the hRAR homolog revealed homology of the predicted protein to the vertebrate steroid receptors as well as to the predicted kni gene product. In situ hybridization of a cDNA probe to wild-type embryos revealed a uniform distribution of apparently maternally-derived transcripts. Zygotic transcript accumulation begins prior to cellular blastoderm in a broad antero-ventral domain. At cellular blastoderm, two additional circumferential bands of transcript appear. These observations suggest that knirps-related (knrl) is an early regulatory gene whose functional activity may be controlled by unidentified ligand.

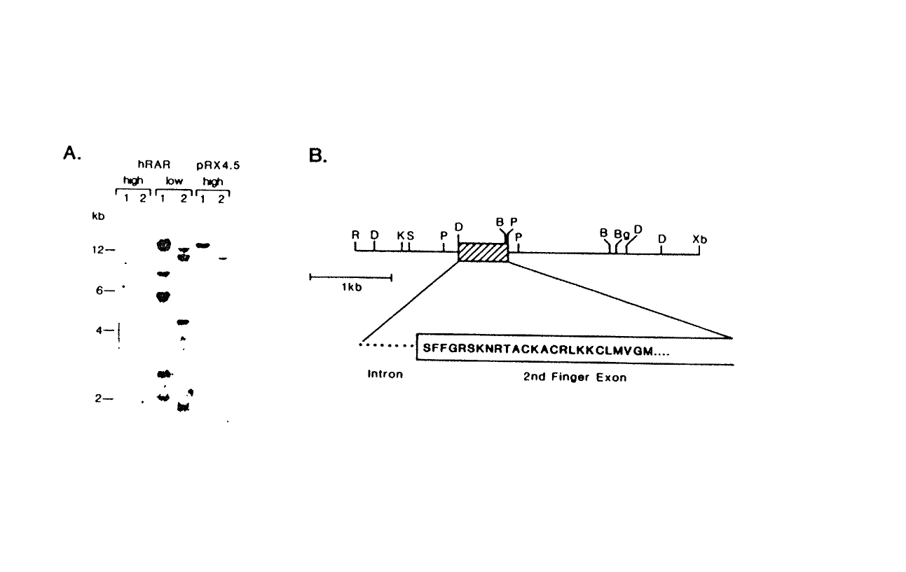

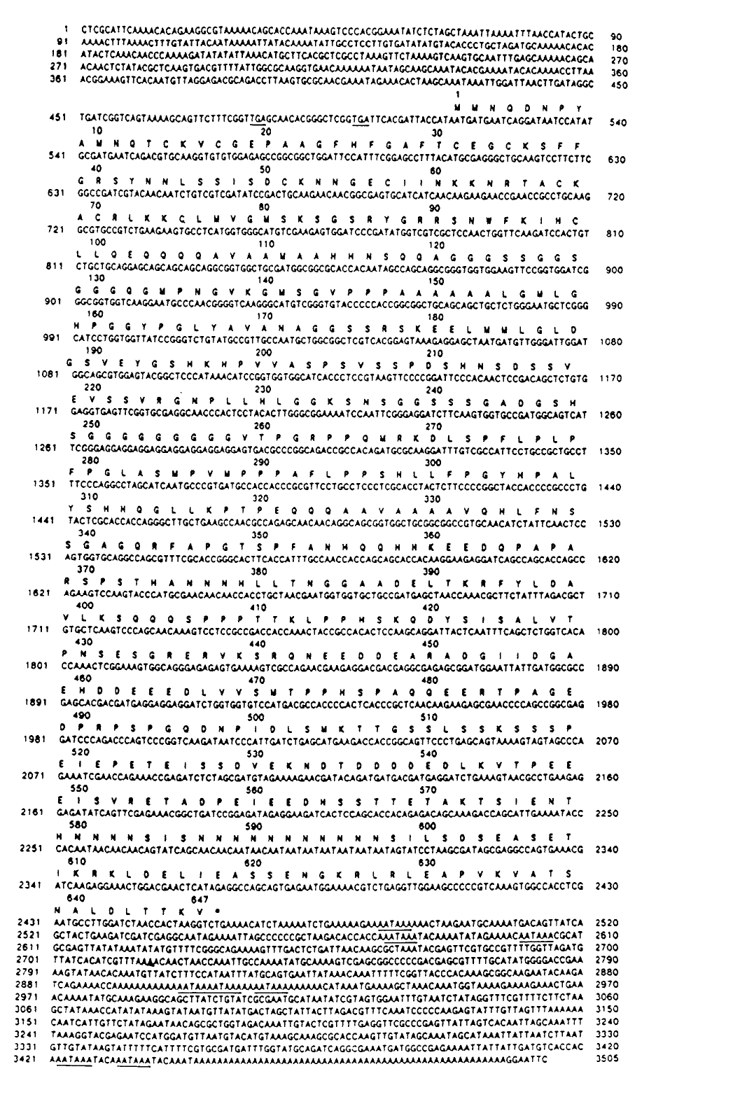

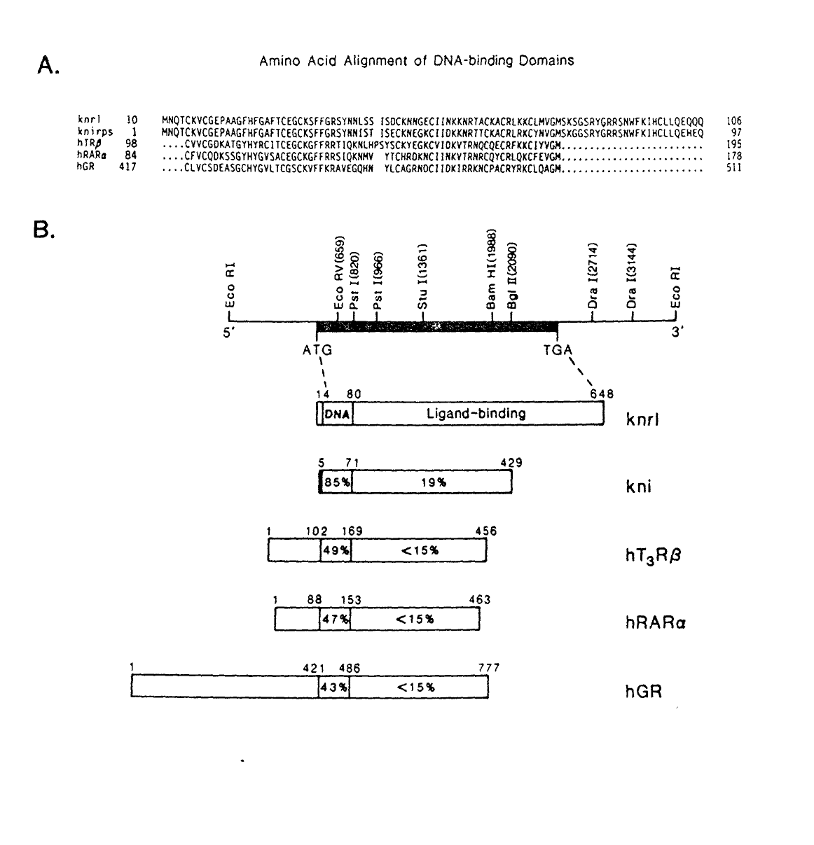

To identify potential homologs of the vertebrate steroid receptors, a Southern blot of Drosophila genomic DNA was probed with a cDNA fragment encoding the hRAR DNA binding domain. Under conditions of reduced hybridization stringency, six distinct EcoRI bands ranging in size from 2 kb to greater than 12 kb were detected (FIG. 1a, lane 1). Screening of a Drosophila genomic library using the same probe and hybridization conditions resulted in the isolation of three classes of inserts (based on cross-hybridization under high stringency conditions). Representatives of each class were hybridized to larval salivary gland polytene chromosomes to identify their cytogenetic location. One class of inserts mapped to 77E 1-2, the same location as the previously identified gap segmentation gene kni. A portion of the genomic insert hybridizing most strongly to the hRAR probe was subcloned and sequenced (plasmid pRX4.5). The derived amino acid sequence for one of the reading frames contained the structural features of a steroid receptor DNA binding domain (FIG. 1b).

To characterize transcripts from the knrl locus, the genomic fragment pRX4.5 was used as a probe to screen a total third instar larval imaginal disc cDNA library. Three cDNA clones were isolated and the complete sequence of one insert, the 3505 base pair (bp) Imd2, is shown in FIG. 2. Imd2 contains an open reading frame capable of encoding 647 amino acids, beginning with the presumptive initiator methionine at nucleotide 1499 and ending with a stop codon beginning at position 2460.

A comparison of the predicted knrl protein with other members of the steroid/thyroid receptor superfamily is shown in FIG. 3. First, sequence alignment demonstrates greatest similarity with the other receptors in the 67 amino acids of the putative knrl DNA-binding domain (FIG. 3A). Between amino acids 14 and 80 of knrl there is 85% amino acid identity with the kni product, 49% with the human thyroid receptor, 47% with the human retinoic acid receptor and 43% with the human glucocorticoid receptor. Interestingly, the knrl and kni DNA binding domains both contain a glycine in the region linking the two zinc fingers (residues 39 and 30 in knrl and kni, respectively), at a position which in all other receptors¹ is either an arginine or lysine. This further suggest a common origin for these two genes. Secondly, the knrl carboxy terminal sequence shows little similarity to those of the other receptors. Structure-function studies with the vertebrate receptors demonstrate that the carboxy terminus contains the ligand binding function and that the relatedness between carboxy termini roughly reflects relatedness of ligand structure. Evans, Science 240, 889 (1988) and Giguere et al., Cell 46, 645 (1986). Therefore, a putative knrl ligand would likely be different from the steroid, retinoid or thyroid hormone classes of ligands.

Analysis of the temporal and spatial expression of knrl suggests that it may function both in early embryogenesis and throughout later development. A Northern blot of stage-specific RNA showed a single RNA species of approximately 3.8 kb expressed at low levels between 0-3 hours after egg-laying (AEL) and at significantly higher levels in later embryos, larvae and adults (data not shown). The spatial location of knrl transcripts was assayed by in situ hybridization using knrl antisense RNa on sections of 0-2 nd 2-4 hour embryos.

The spatially restricted zygotic expression pattern suggests that knrl may be an early regulatory protein. After egg deposition and until approximately the 8th nuclear division, a weak, spatially uniform distribution of apparently maternal transcript was detected. The first apparently zygotic expression is detected at nuclear division 12, when the knrl transcript is localized to a small antero-ventral region of the embryo, at approximately 80-100% of egg length (EL) on the ventral side (domain I). Expression in this domain intensifies through the cellular blastoderm stage, and two additional circumferential bands of transcript become detectable, centered at approximately 70% EL ventrally (domain II) and 25% EL ventrally (domain III). It is noteworthy that expression in domain II appears significantly more intense ventrally than dorsally.

Based on its spatial and temporal patterns of expression and the well-characterized role steroid hormone receptors play in transcriptional regulation, knrl is a candidate for an early regulatory gene.

The homology of the predicted knrl gene product to vertebrate steroid receptors suggests that its function is ligand-dependent. If this is the case, such a ligand might constitute a previously unrecognized small-molecule morphogen, and some of the genes involved in regulating knrl function might affect the synthesis of the ligand or storage of a ligand precursor, rather than regulating knrl expression. However, the unrelatedness of the knrl carboxy terminus to that of the other receptors makes it difficult to predict a potential ligand. Regardless, knrl is a new member of the steroid receptor gene family, whose products contribute to morphogenesis and pattern formation in both vertebrates and invertebrates.

The foregoing description details specific methods that can be employed to practice the present invention. Having detailed the specific methods initially used to identify and isolate particular model Drosophila receptors hereof, as to protein and DNA sequences, characterization and use, the art skilled will well enough know how to devise alternative, reliable methods for arriving at the same information and for extending this information to other insect receptors and other related polypeptides. Thus, however detailed the foregoing may appear in text, it should not be construed as limiting the overall scope hereof; rather, the ambit of the present invention is to be governed only by the lawful construction of the appended claims.

1. An insect receptor polypeptide, having a sequence characteristic of a mammalian receptor DNA-binding binding domain having flanking N-terminal and C-terminal sequences, and having purity sufficient to provide sufficient coding sequence to enable the production of total DNA coding sequence of said receptor or cross-hybridizing hybridizing DNA of related receptors for use in the expression thereof in recombinant host cells operatively transfected with said DNA.

2. The insect receptor according to claim 1 of a sequence as set forth in FIG. 2 being a part hereof including modifications of such sequence resulting in an insect receptor having requisite in kind biofunction.

3. The insect receptor according to claim 2 wherein said modification is the result of the substitution, addition, deletion or inversion of one or more amino acids along the backbone chain.

4. The insect receptor according to claim 1 which is a Drosophila melanogaster knirps-related polypeptide.

5. The insect receptor according to claim 1 wherein said DNA hybridizes with corresponding DNA sequences of the same or other species under stringent hybridization conditions.

6. The insect receptor according to claim 1 being a DNA cross-hybridizable, related receptor.

7. A DNA molecule that is a recombinant DNA molecule or a cDNA molecule consisting of a sequence encoding an insect receptor polypeptide or a functional fragment thereof.

8. A DNA molecule according to claim 7 comprising the DNA-binding sequence of said insect receptor.

9. A DNA molecule according to claim 8 comprising additionally the trans-activation transcription domain of said insect receptor.

10. An expression vector operatively harboring a DNA molecule according to any one of claim 7, 8 or 9.

11. A recombinant host cell transfected with an expression vector according to claim 10.

12. A recombinant host cell according to claim 11 which is an E. coli strain.

13. A recombinant host cell according to claim 11 which is an eukaryotic cell.

14. A cell culture comprising cells according to claim 11 and an extrinsic support medium assuring the viability of said cells.

15. A process which comprises the preparation of a polypeptide according to claim 1, wherein the polypeptide is prepared by expression in a recombinant host cell of transfecting DNA encoding said polypeptide.

16. The process according to claim 15 which includes recovering and purifying the polypeptide.

17. A process which, following the preparation of a polypeptide having insect receptor biofunction by a process according to claim 15 or 16, comprising the use of said polypeptide in the manufacture of a composition useful for administration to a subject.

18. An assay for screening and identifying materials having a putative potential of modulating the biofunction of an insect receptor comprising the steps comprising:
a) providing an insect receptor species,
b) challenging said insect receptor species with one or more of a battery of test materials having putative potential of modulating the biofunction of an insect receptor,
c) monitoring the effect of said test material by measuring the amount of transcription induced by said receptor species, and
d) selecting candidates from the battery of tested materials.

19. An assay according to claim 18 further comprising the additional step following d), comprising:
e) employing said candidate in the preparation of a composition containing said candidate as an essential component, said composition being useful to impart its biofunction properties on a corresponding insect receptor when it is contacted in vivo with said receptor.

20. An assay according to claim 19 further comprising the additional step following step e), comprising:
f) contacting said composition with an insect.

21. An assay according to any one of the preceding claim 19 or 20 wherein said composition is an insecticide.
